(19) United States
(12) Patent Application Publication
(10) Pub. No.: US 2003/0003085 A1
(43) Pub. Date: Jan. 2, 2003

(54) Muscle-derived stem cells and uses therefor

(75) Inventors: Louis M. Kunkel, Westwood, MA (US); Emanuela Gussoni, Winchester, MA (US); Richard C. Mulligan, Lincoln, MA (US); Yuko Soneoka, Washington, DC (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Children's Medical Center Corporation, Boston, MA

(21) Appl. No.: 10/097,190

(22) Filed: Mar. 13, 2002

Related U.S. Application Data
(63) Continuation of application No. PCT/US00/25129, filed on Sep. 14, 2000.
(60) Provisional application No. 60/153,822, filed on Sep. 14, 1999.

(51) Int. Cl. A61K 48/00

(52) U.S. Cl. 424/93.21; 424/93.7

(57) ABSTRACT

A method for purifying muscle stem cells from a myoblast sample isolated from mammalian skeletal muscle is disclosed. Purified muscle stem cells can be used for a variety of purposes, including for systemic delivery of muscle proteins and other desired nucleic acid products to a mammal, for gene therapy, in the treatment muscle diseases, including muscular dystrophies, in the treatment or prophylaxis of inherited or acquired diseases, including genetic diseases and cancer, and in transplanting bone marrow to a mammal.

### RELATED APPLICATIONS

This application is a continuation of International Application No. PCT/US00/25129, filed Sep. 14, 2000, which claims the benefit of U.S. Provisional Application No. 60/153,822, filed Sep. 14, 1999. The teachings of the above applications are incorporated herein by reference in their entirety.

### BACKGROUND OF THE INVENTION

Muscle precursor cells (myoblasts) are thought to be stem cells of skeletal muscle capable of repairing damaged or injured myofibers (Mauro, A., J. Biophys. Biochem. Cytol., 9:493-495 (1961); Bischoff, R., in Myology, Engel, A. G. and Franzini-Armstrong, C., Eds., New York: McGraw Hill, pp. 97-119, 1994; and Grounds, M., Adv. Exp. Med. Biol., 280:101-104 (1990)). Because myoblasts are thought to be capable of repairing damaged or injured myofibers, the technique of myoblast transfer (myoblast transplantation) has been proposed as a potential therapy or cure for muscular diseases, including Duchenne muscular dystropy (DMD).

Myoblast transfer involves injecting myoblast cells into the muscle of a mammal, particularly a human patient, requiring treatment. Although developed muscle fibers are not regenerative, the myoblasts are capable of a limited amount of proliferation, thus increasing the number of muscle cells at the location of myoblast infusion. Myoblasts so transferred into mature muscle tissue will proliferate and differentiate into mature muscle fibers. This process involves the fusion of mononucleated myogenic cells (myoblasts) to form a multinucleated syncytium (myofiber or myotube). Thus, it has been proposed that muscle tissue which has been compromised either by disease or trauma may be supplemented by the transfer of myoblasts into the compromised tissue.

Dystrophin is a muscle-specific protein that is localized on the plasma membrane of all muscle cells and is responsible for maintaining cellular integrity during muscle contractions (Hoffman et al. Cell, 51:919-928 (1987); Koenig et al., Cell, 53:219-228 (1988); and Watkins et al., Nature, 333:863-866 (1988)). It has been shown that myoblasts injected into genetically deficient X-linked muscular dystrophic (mdx) mice fuse into the muscle fibers of the host, and are capable of expressing a recombinant gene product, dystrophin (an intracellular protein, the lack of which causes Duchenne muscular dystrophy (DMD)), although at inefficient levels (Karpati, G. et al., Am. J. Pathol., 135:27-32 (1989); and Partridge, T. A. et al., Nature, 337:176-179 (1989)). Human clinical trials have also revealed minimal expression of normal donor dystrophin in patients injected with normal donor myoblasts (Law, P. K. et al., Lancet, 336:114-115 (1990); Mendell, J. R. et al., N. Engl. J. Med., 333:832-838 (1995); Morandi, L. et al., Neuromuscul. Disord., 5:291-295 (1995); Gussoni, E. et al., Nature, 356:435-438 (1992); Huard, J. et al., Muscle Nerve, 15:550-560 (1992); Karpati, G. et al., Ann. Neurol., 34:8-17 (1993); and Neumeyer, A. M. et al., Neurology, 51:589-592 (1998)).

However, although myoblast transfer has shown a great potential utility, that utility is limited by inefficient expression of donor dystrophin in recipients, possibly due to rapid death of introduced donor myoblasts (Fan, Y. et al., Muscle Nerve, 19:853-860 (1996)), lack of migration of donor myoblasts from the site of injection, and the need to deliver donor cells directly to muscle by multiple, local intramuscular injections. Clearly, there is considerable interest in developing alternative approaches for therapy of muscular diseases which overcome the current limitations of myoblast transfer methods.

### SUMMARY OF THE INVENTION

The present invention relates to the discovery of two populations of muscle cells in myoblast samples isolated from mammalian skeletal muscle: side population (SP) cells and main population (MP) cells. Muscle SP cells are also referred to herein as muscle stem cells.

The present invention provides a method of purifying or isolating muscle stem cells from a myoblast sample isolated from mammalian skeletal muscle. The method comprises combining the myoblast sample with a fluorescent, lipophilic vital dye which is a substrate for a multidrug resistance protein under conditions appropriate for uptake of the dye by cells in the myoblast sample. As used herein, the term “substrate” refers to a substance which is removed from the cell by the multidrug resistance protein. The term “multidrug resistance protein”, as used herein, includes the multidrug resistance protein and multidrug resistance-like proteins, which are proteins that exhibit multidrug resistance-like activity (i.e., a multidrug resistance protein-like efflux of a dye from muscle SP cells). For example, the term “multidrug resistance protein” includes analogs and derivatives of the multidrug resistance protein. The resulting combination is exposed to an excitation wavelength which results in fluorescence of the dye, which is observed (assessed) at an emission wavelength. The amount of dye contained (exhibited) by each cell population resolved at the emission wavelength is analyzed (observed). The population of nucleated cells which contains the lowest amount of dye at the emission wavelength, relative to the other population of nucleated cells, is isolated. The population of nucleated cells which contains the lowest amount of dye at the emission wavelength, relative to the other population of nucleated cells, is the muscle stem cells.

The present invention also provides a method for separating muscle stem cells from muscle MP cells in a myoblast sample isolated from mammalian skeletal muscle. This method comprises combining the myoblast sample with a fluorescent, lipophilic vital dye which is a substrate for a multidrug resistance protein under conditions appropriate for uptake of the dye by cells in the myoblast sample, and exposing the resulting combination to an excitation wavelength which results in fluorescence of the dye, which is observed (assessed) at an emission wavelength. The amount of dye exhibited by each cell population resolved at the emission wavelength is analyzed. The population of nucleated cells which contains the lowest amount of dye at the emission wavelength is isolated. The population of nucleated cells which contains the lowest amount of dye at the emission wavelength, relative to the other population of nucleated cells, is the muscle stem cells. The population of nucleated cells which contains a greater amount of dye at the emission wavelength, relative to the other population of nucleated cells, is the muscle MP cells.

In a preferred embodiment, a dye which does not affect the staining profile of the fluorescent vital dye, but which allows exclusion of dead cells, is added in addition to the fluorescent vital dye.

Typically, as a negative control, a myoblast sample isolated from mammalian skeletal muscle is stained with a fluorescent vital dye in the presence of an inhibitor of a multidrug resistance protein. Simultaneously, as the test sample, a second myoblast sample is stained with the fluorescent vital dye in the absence of the inhibitor. The stained samples (negative control and test sample) are exposed to an excitation wavelength which results in fluorescence of the dye, which is assessed (observed) at an emission wavelength. The cell populations observed in the negative control are compared with the cell populations observed in the test sample. Muscle stem cells will be visible in the test sample but will not be visible in the negative control. This approach can be used to define the location (set the gate) for isolation of muscle stem cells using the methods of the present invention.

The invention also relates to muscle stem cells purified using or obtainable by (obtained by) the methods described herein. In one embodiment, the purified muscle stem cells of the present invention are Sca-1^{pos} lin^{neg}, c-kit^{neg} and CD45^{neg}. In another embodiment, the purified muscle stem cells are also CD43^{neg}. In a preferred embodiment, the purified muscle stem cells are isolated from human muscle tissue.

Muscle stem cells purified or isolated using the methods of the present invention can be introduced systemically into individuals where these stem cells are capable of (1) reconstituting the bone marrow of lethally irradiated individuals and (2) migrating to the skeletal muscle of recipient individuals and expressing protein(s) normally missing in these individuals. Purified muscle stem cells of the present invention can be used in systemic delivery of muscle proteins and recombinant non-muscle proteins or other desired nucleic acid products in the treatment of a number of acquired and inherited human diseases. Gene therapy using purified muscle stem cells of the present invention can be applied in providing essential gene products to muscle tissue and to the circulation through secretion from muscle tissue. The purified muscle stem cells used can be obtained from a mammal to whom they will be returned or from another/different mammal of the same or different species (donor) and introduced into a recipient mammal.

Thus, the invention also relates to a method for delivery of a muscle protein to the circulation of a mammal (e.g., a human or other mammal or vertebrate) comprising administering an effective amount of purified donor muscle stem cells of the present invention to the mammal. A muscle protein, as used herein, refers to a protein which, when defective or absent in a patient, is responsible for a particular muscle disease or disorder. In a particular embodiment, the muscle protein is dystrophin. Other muscle proteins include calpain-3, sarcoglycan complex members (e.g., α-sarcoglycan, β-sarcoglycan, γ-sarcoglycan and δ-sarcoglycan) and laminin α2-chain.

The muscle stem cells can be used in delivery of a muscle protein for treatment of muscle diseases or disorders, such as muscular dystrophies, in a mammal in need of such treatment. Muscular dystrophies include Duchenne muscular dystrophy (DMD) Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies and Emery-Dreifuss muscular dystrophy. As a result, the invention also relates to a method of treating a muscle disease or disorder in a mammal (e.g., a human or other mammal or vertebrate) in need thereof comprising administering an effective amount of purified donor muscle stem cells to the mammal. In a particular embodiment, the muscle disease or disorder is a muscular dystrophy, such as DMD or BMD. In mammals with DMD or BMD, a proportion of the administered donor muscle SP cells can fuse with DMD or BMD host muscle fibers, contributing dystrophin-competent myonuclei to the host fibers (mosaic fibers). The expression of normal (donor) dystrophin genes in such fibers can generate sufficient dystrophin in some segments to confer a normal phenotype to these muscle fiber segments. In another embodiment, the muscle disease or disorder is a limb-girdle muscular dystrophy.

The invention further relates to a method for delivery of a desired nucleic acid product to the circulation of a mammal (e.g., a human or other mammal or vertebrate) comprising (a) introducing a nucleic acid sequence encoding the desired nucleic acid product into purified donor muscle stem cells of the present invention, thereby producing recombinant muscle stem cells; and (b) administering to the mammal the recombinant muscle stem cells produced in step (a). A desired nucleic acid product, as used herein, refers to the desired protein or polypetide, DNA or RNA (e.g., gene product) to be expressed in the mammal. In a particular embodiment, the desired nucleic acid product is a heterologous therapeutic protein.

Generally, a nucleic acid sequence encoding a desired nucleic acid product will be introduced into muscle stem cells of the present invention through the use of viral vectors, such as DNA or RNA (retroviral) vectors. Retroviruses have been shown to have properties which make them particularly well suited to serve as recombinant vectors by which a nucleic acid sequence encoding a desired nucleic acid product can be introduced into mammalian (e.g., a human or other mammal or vertebrate) cells. For example, recombinant retrovirus for use in delivery of a desired nucleic acid product can be generated by introducing a suitable proviral DNA vector encoding the desired nucleic acid product into fibroblastic cells that produce the viral proteins necessary for encapsidation of the desired recombinant RNA. This is one approach which can be used to introduce (deliver) a nucleic acid sequence encoding a desired nucleic acid product into muscle stem cells of the present invention for delivery of the desired nucleic acid product to the circulation of a mammal. See, for example, Mann, R. et al., Cell, 33:153-159 (1983); Watanabe, S. and H. M. Temin, Mol. Cell. Biol., 3:2241-2249 (1983); Cone, R. D. and R. C. Mulligan, Proc. Natl. Acad. Sci. USA, 81:6349-6353 (1984); Soneoka, Y. et al., Nucl. Acids Research, 123:628-633 (1995); and Danos, O. and R. C. Mulligan, U.S. Pat. No. 5,449,614.

The invention also relates to a method of in vivo administration of a desired nucleic acid product to a mammal comprising infecting or transfecting purified donor muscle stem cells with a viral vector comprising a nucleic acid sequence encoding the desired nucleic acid product and introducing the infected or transfected purified donor muscle stem cells into the mammal, in which the desired nucleic acid product is expressed.

The present invention further relates to a method of transplanting bone marrow in a mammal comprising introducing into the mammal purified muscle stem cells. Purified muscle stem cells of the present invention can be used to treat diseases or conditions in which a mammal needs bone marrow cells (e.g., leukemia, thalassemia and anemia).

The muscle stem cells can be used in delivery of a desired nucleic acid product for the treatment or prophylaxis of inherited or acquired diseases (e.g., genetic diseases) in a mammal in need of such treatment. As a result, the invention also relates to a method of treating or prophylaxis of an inherited acquired disease (e.g., a genetic disease) in a mammal in need thereof comprising (a) introducing a nucleic acid sequence encoding a desired nucleic acid product into purified donor muscle stem cells of the present invention, thereby producing recombinant muscle stem cells; and (b) administering to the mammal the recombinant muscle stem cells produced in step (a). In a particular embodiment, the nucleic acid sequence encoding the desired nucleic acid product is incorporated into a viral vector.

The invention also provides a method for treating or prophylaxis of a cancer in a mammal in need thereof comprising (a) introducing a nucleic acid sequence encoding a desired anticancer agent into purified donor muscle stem cells of the invention, thereby producing recombinant muscle stem cells; and (b) administering to the mammal the recombinant muscle stem cells produced in step (a). In a particular embodiment, the nucleic acid sequence encoding the desired anticancer agent is incorporated into a viral vector.

The invention further provides uses of muscle stem cells for the manufacture of medicaments for use in the treatment or prophylaxis of a cancer, a genetic disease, an inherited or acquired disease, or a muscle disease in a mammal. In a particular embodiment, a nucleic acid sequence encoding a desired nucleic acid product is introduced into the muscle stem cells.

The invention also relates to muscle stem cells that are used in the methods and uses described herein.

### DETAILED DESCRIPTION OF THE INVENTION

The present invention relates to the use of fluorescence-activated cell sorter (FACS) analysis and sorting of muscle cells stained with a fluorescent vital dye to purify or isolate muscle SP cells from a myoblast sample isolated from mammalian skeletal muscle. The present invention also relates to the use of FACS analysis and sorting of muscle cells stained with a fluorescent vital dye to separate muscle SP cells from muscle MP cells. Two populations of nucleated muscle cells are revealed by FACS analysis of myoblast samples isolated from mammalian skeletal muscle and stained with a fluorescent vital dye in accordance with the present invention. The population of nucleated cells which displays low staining with the dye (i.e., contains (exhibits) the smaller or lower amount of dye), relative to the other population of nucleated cells, is muscle SP cells. Muscle SP cells are also referred to herein as muscle stem cells. The population of nucleated cells which is more brightly stained with the dye (i.e., contains (exhibits) the greater or higher amount of dye), relative to the other population of nucleated cells, is muscle MP cells.

The intensity of staining of a population of nucleated muscle cells in a myoblast sample (i.e., the amount of dye present in a population of nucleated muscle cells), relative to the other population of nucleated muscle cells in the myoblast sample, is observed (analyzed) for differences in intensity of fluorescence. This information is used to define the area in the sort profile where the muscle stem cells reside.

FACS sorting is used to isolate (purify) the population of nucleated cells which contains the lowest amount of dye at the emission wavelength relative to the other population of nucleated cells, resulting in purified or isolated muscle stem cells.

The muscle stem cell purification and separation strategies described herein can be applied to myoblast samples isolated from skeletal muscle of any mammalian species. The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminents (e.g., cows, pigs, horses).

Myoblast samples used in the muscle stem cell purification and separation methods of the present invention can be isolated from the skeletal muscle of any mammal according to methods generally known in the art. For example, myoblast samples can be isolated from muscle biopsies using standard culture techniques as described in, for example, Blau, H. M. et al., Adv. Exp. Med. Biol., 280:97-100 (1990); Blau, H. M. et al., Proc. Natl. Acad. Sci. USA, 78:5623-5627 (1981); and Rando, T. A. and Blau, H. M., J. Cell Biol., 125:1275-1287 (1994), the teachings of which are incorporated herein by reference. See also, e.g., Webster, C. et al., Exp. Cell Res., 174:252-265 (1988); Gussoni, E. et al., Nature, 356:435-438 (1992); Karpati, G. et al., Ann. Neurol., 34:8-17 (1993); Walsh, F. A. et al., Adv. Exp. Med. Biol., 28:41-46 (1990); Ham, R. G. et al., Adv. Exp. Med. Biol., 280:193-199 (1990); and Morgan, J. E. et al., J. Neurol. Sci., 86:137-147 (1988). Myoblast samples used in the muscle stem cell purification and separation methods of the present invention typically comprise about 10⁴ to 10⁸ cells, and preferably, about 10⁶ cells. Myoblasts samples used in the muscle stem cell purification and separation methods of the present invention can also comprise more than 10⁸ cells.

The fluorescent vital dye which can be used in the present invention is a substrate for a multidrug resistance protein. Preferably, the dye is also lipophilic. The term “substrate”, as used herein, refers to a substance which is removed from the cell by the multidrug resistance protein. The term “multidrug resistance protein”, as used herein, includes the multidrug resistance protein and multidrug resistance-like proteins, which are proteins that exhibit multidrug resistance-like activity (i.e., a multidrug resistance protein-like efflux of a dye from muscle SP cells). For example, the term “multidrug resistance protein” includes analogs and derivatives of the multidrug resistance protein. Suitable dyes for use in the present invention are well known in the art. In a preferred embodiment, the vital dye used is Hoechst 33342 (H0342), a fluorescent dye which is readily taken up by live cells. In an alternative embodiment, the vital dye used is Rhodamine 123.

A dye which does not affect the staining profile of the fluorescent vital dye, but which allows exclusion of dead cells, can be added in addition to the fluorescent vital dye. Preferably, a dye which allows exclusion of dead cells is added after staining of the myoblast sample with the fluorescent vital dye. An example of a dye which allows exclusion of dead cells is propidium iodine (PI). Other suitable dyes that can be used to exclude dead cells are known in the art.

Suitable excitation wavelengths used in the present invention are those which will excite the particular dye employed to a measurable extent. For example, in the embodiment in which Hoechst 33342 is employed as the vital dye, an appropriate excitation wavelength is from about 250 nm to about 450 nm, and in a particular embodiment, is at about 350 nm.

Fluorescence of the dye can be observed (assessed) at one emission wavelength. Alternatively, fluorescence of the dye can be observed (assessed) at two emission wavelengths. Suitable emission wavelengths are those which will measure fluorescence of the dye employed in the methods of the present invention so that distinct populations of live muscle cells are resolved as a result of the differences in intensity of fluorescence. For example, Hoechst 33342 emission can be detected at a range of wavelengths, from about 400 nm to about 700 nm, and in a particular embodiment, about 600 nm. Hoechst 33342 emission can also be detected at simultaneous wavelengths of about 450 nm and about 650 nm. Hoechst 33342 emission is detected with a 400 nm long pass filter. Propidium iodide fluorescence is detected with a 610 nm long pass filter.

The amount of dye used in the muscle stem cell purification and separation methods of the present invention will be an amount which is sufficient to stain the cells. The amount of dye will vary depending on the particular dye employed and the source of the cells. In a particular embodiment, the amount of dye used is from about 5 μg/ml to about 20 μg/ml dye, preferably from about 10 μg/ml to about 15 μg/ml dye, and in a more particular embodiment, about 12.5 μg/ml dye. In a second embodiment, the amount of dye used is generally about 2.5 times greater than the amount of dye used to purify bone marrow SP cells (Goodell, M. A. et al., J. Exp. Med., 183:1797-1806 (1996)). In a third embodiment, the amount of dye used is from about 1 μg/ml to about 5 μg/ml dye.

The staining time with the dye (i.e., the length of time cells are exposed to dye) varies depending on the temperature at which staining is to occur and the dye concentration used. Thus, staining can occur overnight or over a number of days at the appropriate temperature. In particular, the staining time with the dye can be from about 30 minutes to about 180 minutes, preferably between about 60 minutes to about 120 minutes. In a particular embodiment, the staining time with Hoechst 33342 is about 90 minutes. In a further embodiment, the staining time with Hoechst 33342 is about 60 minutes.

The temperature at which staining with the dye can be carried out is from about 4° C. to about 45° C., preferably about 15° C. to about 45° C., and in particular, about 37° C.

In a particular embodiment of the method of purifying or isolating muscle SP cells of the present invention, a myoblast sample isolated from mammalian skeletal muscle is stained with 12.5 μg of Hoechst 33342 for 90 minutes at 37° C. In another embodiment, a myoblast sample isolated from mammalian skeletal muscle is stained with 5 μg of Hoechst 33342 for 90 minutes at 37° C. In a third embodiment, a myoblast sample isolated from mammalian skeletal muscle is stained with 5 μg of Hoechst 33342 for 60 minutes at 37° C.

Muscle SP cells stained with a fluorescent vital dye in the presence of an inhibitor of a multidrug resistance protein are not visible. In contrast, muscle MP cells stained with a fluorescent vital dye in the presence of an inhibitor of a multidrug resistance protein are visible. Thus, myoblast samples stained with a fluorescent vital dye in the presence of an inhibitor of a multidrug resistance protein can be used as a negative control for muscle SP cells. In addition, myoblast samples stained with a fluorescent vital dye in the presence of an inhibitor of a multidrug resistance protein can be utilized to set the gate for isolation of muscle SP cells by FACS sorting in a test sample. It is important to set the gate to define which cells are to be purified or isolated by FACS sorting. As used herein, “to set the gate” means to define the area in the sort profile where SP cells reside in order to purify them. A “test sample” is a myoblast sample from which muscle SP cells are purified or isolated using the methods of the present invention. An “inhibitor of a multidrug resistance protein”, as defined herein, is a substance or agent which inhibits or interferes with the activity of the multidrug resistance protein expressed by SP cells. More specifically, an inhibitor of a multidrug resistance protein is a substance or agent which interferes with the ability of the multidrug resistance protein to remove dye from muscle SP cells. Inhibitors of the multidrug resistance protein include verapamil, antibodies directed against multidrug resistance protein (i.e., anti-multidrug resistance protein antibody), reserpine, PAK-104P, vincristine and SDZ PSC 833.

As a particular example, to set the gate for isolation of muscle SP cells in a test sample, a myoblast sample isolated from mammalian skeletal muscle is stained with a fluorescent vital dye in the presence of an inhibitor of a multidrug resistance protein (negative control). Simultaneously, a second myoblast sample (test sample) is stained with the fluorescent vital dye in the absence of the inhibitor. The stained samples (negative control and test sample) are exposed to an excitation wavelength which results in fluorescence of the dye, which is assessed (observed) at an emission wavelength. The cell populations observed in the negative control are compared with the cell populations observed in the test sample. Muscle SP cells are visible in the test sample but are not visible in the negative control. This information can be used to define the area in the sort profile where the muscle SP cells reside in the test sample for isolation.

Thus, the present invention provides a method of purifying or isolating muscle SP cells from a myoblast sample isolated from mammalian skeletal muscle comprising combining the myoblast sample with a fluorescent, lipophilic vital dye which is a substrate for a multidrug resistance protein under conditions appropriate for uptake of the dye by cells in the myoblast sample. The resulting combination is exposed to an excitation wavelength which results in fluorescence of the dye, which is assessed (observed) at an emission wavelength. The amount of dye contained (exhibited) by each cell population resolved at the emission wavelength is analyzed. The population of nucleated cells which contains (exhibits) the lowest amount of dye at the emission wavelength, relative to the other population of nucleated cells, is isolated (purified). The population of nucleated cells which contains (exhibits) the lowest amount of dye at the emission wavelength, relative to the other population of nucleated cells, is muscle SP cells.

The present invention also provides a method for separating muscle SP cells from muscle MP cells in a myoblast sample isolated from mammalian skeletal muscle comprising combining the myoblast sample with a fluorescent, lipophilic vital dye which is a substrate for a multidrug resistance protein under conditions appropriate for uptake of the dye by cells in the myoblast sample, and exposing the resulting combination to an excitation wavelength which results in fluorescence of the dye, which is observed (assessed) at an emission wavelength. The amount of dye exhibited by each cell population at the emission wavelength is analyzed. The population of nucleated cells which contains (exhibits) the lowest amount of dye at the emission wavelength is isolated (purified). The population of nucleated cells which contains (exhibits) the lowest amount of dye at the emission wavelength, relative to the other population of nucleated cells, is the muscle SP cells. The population of nucleated cells which contains (exhibits) a greater amount of dye at the emission wavelength, relative to the other population of nucleated cells, is the muscle MP cells.

Characterization of murine muscle SP cells purified using the muscle stem cell purification method of the present invention revealed them to be Sca-1^{pos} lin^{neg}, c-kit^{neg} and CD45^{neg}. In addition, over 90% of the murine muscle SP cells were found to be negative for CD43. In contrast, the murine muscle MP cells expressed some lineage markers (lin^{pos}), such as CD11, Gr-1 and CD5. In addition, as described herein, Applicants have discovered that muscle SP cells in culture maintain their spherical shape and do not adhere to the plate. In contrast, muscle MP cells under the same experimental conditions are morphologically differentiated. These results indicate that muscle SP cells are derived from a less differentiated progenitor population than the MP cells and that muscle SP cells are less differentiated than muscle MP cells.

Characterization of human muscle SP cells purified using the muscle stem cell purification method of the present invention revealed them to be c-kit^{neg} and CD123^{neg}. Some human muscle SP cells were found to be positive for AC133 (Miraglia, S. et al., Blood, 90:5013-5021 (1997); Yin, A. H. et al., Blood, 90:5002-5012 (1997)), while others were found to be negative for AC133. Similarly, some human muscle SP cells were found to be positive for CD34, while others were found to be negative for CD34. In addition, some human muscle SP cells were found to be positive for CD90, while others were found to be negative for CD90.

As described herein, Applicants have also discovered that muscle stem cells isolated from skeletal muscle have the potential to divide in vivo and fuse into host muscle. Specifically, by injecting 10,000-20,000 muscle SP cells into the circulation, up to 9% of dystrophin-positive myofibers have been detected in the skeletal muscles of mdx animals, indicating that muscle SP cells are successfully recruited from the circulation into skeletal muscles where they are able to fuse with preexisting myofibers and express dystrophin. This percentage is similar to what has been previously seen after intramuscular injection of 5×10⁵ primary myoblasts in individual muscles (Karpati, G. et al., Am. J. Pathol., 135:27-32 (1989); Fan, Y. et al., Muscle Nerve, 19:853-860 (1996); and Beauchamp, J. et al., Muscle Nerve, Supplement 1: S261 (1994)).

As described herein, muscle SP cells have been shown to protect recipients from the consequences of lethal irradiation. That is, muscle SP cells have been shown to allow survival of lethally irradiated recipients. Surprisingly, Applicants have also discovered that muscle SP cells can be used to successfully repopulate stem cell activity in lethally irradiated recipients. Moreover, donor muscle SP cells can be found in the bone marrow and spleen as differentiated hematopoietic cells, thereby suggesting that muscle SP cells have the ability to differentiate into a variety of mesodermal tissues under the influence of the local environment.

Thus, muscle SP cells purified or isolated using the methods of the present invention can be used for delivery of a muscle protein to the circulation of a mammal. A muscle protein, as used herein, refers to a protein which, when defective or absent in a mammal, is responsible for a particular muscle disease or disorder. In a particular embodiment, the muscle protein is dystrophin. Other muscle proteins include calpain-3, sarcoglycan complex members (e.g., α-sarcoglycan, β-sarcoglycan, γ-sarcoglycan and δ-sarcoglycan) and laminin α2-chain. The term circulation is meant to refer to blood circulation. The term blood refers to the “circulating tissue” of the body, the fluid and its suspended formed elements that are circulated through the heart, arteries, capillaries and veins.

In the method for delivery of a muscle protein to the circulation of a mammal, an effective amount of purified donor muscle SP cells is transplanted into a mammal in need of such treatment (also referred to as a recipient or a recipient mammal). As used herein, “donor” refers to a mammal that is the natural source of the purified muscle SP cells. Preferably, the donor is a healthy mammal (e.g., a mammal that is not suffering from a muscle disease or disorder). In a particular embodiment, the donor and recipient are matched for immunocompatibility. Preferably, the donor and the recipient are matched for their compatibility for the major histocompatibility complex (MHC) (human leukocyte antigen (HLA))-class I (e.g., loci A,B,C) and -class II (e.g., loci DR, DQ, DRW) antigens. Immunocompatibility between donor and recipient are determined according to methods generally known in the art (see, e.g., Charron, D. J., Curr. Opin. Hematol., 3:416-422 (1996); Goldman, J., Curr. Opin. Hematol., 5:417-418 (1998); and Boisjoly, H. M. et al., Opthalmology, 93:1290-1297 (1986)). In an embodiment of particular interest, the recipient a human patient.

As used herein, muscle diseases and disorders include, but are not limited to, recessive or inherited myopathies, such as, but not limited to, muscular dystrophies. Muscular dystrophies are genetic diseases characterized by progressive weakness and degeneration of the skeletal or voluntary muscles which control movement. The muscles of the heart and some other involuntary muscles are also affected in some forms of muscular dystrophy. The histologic picture shows variation in fiber size, muscle cell necrosis and regeneration, and often proliferation of connective and adipose tissue. Muscular dystrophies are described in the art and include Duchenne muscular dystrophy (DMD), Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies and Emery-Dreifuss muscular dystrophy. See, e.g., Hoffman et al., N. Engl. J. Med., 318:1363-1368 (1988); Bönnemann, C. G. et al., Curr. Opin. Ped., 8:569-582 (1996); Worton, R., Science, 270:755-756 (1995); Funakoshi, M. et al., Neuromuscul. Disord., 9(2):108-114 (1999); Lim, L. E. and Campbell, K. P., Curr. Opin. Neurol., 11(5):443-452 (1998); Voit, T., Brain Dev., 20(2):65-74 (1998); Brown, R. H., Annu. Rev. Med., 48:457-466 (1997); Fisher, J. and Upadhyaya, M., Neuromuscul. Disord., 7(1):55-62 (1997), which references are incorporated entirely incorporated herein by reference.

Two major types of muscular dystrophy, DMD and BMD, are allelic, lethal degenerative muscle diseases. DMD results from mutations in the dystrophin gene on the X-chromosome (Hoffman et al., N. Engl. J. Med., 318:1363-1368 (1988)), which usually result in the absence of dystrophin, a cytoskeletal protein in skeletal and cardiac muscle. BMD is the result of mutations in the same gene (Hoffman et al., N. Engl. J. Med., 318:1363-1368 (1988)), but dystrophin is usually expressed in muscle but at a reduced level and/or as a shorter, internally deleted form, resulting in a milder phenotype.

Thus, the present invention also provides a method of treating a muscle disease or disorder in a mammal in need thereof comprising administering an effective amount of purified donor muscle SP cells to the mammal. In a particular embodiment, the invention relates to a method of treating a muscular dystrophy in a mammal in need thereof comprising administering an effective amount of purified donor muscle SP cells to the mammal. In another embodiment, the invention relates to a method of treating DMD in a mammal in need thereof comprising administering an effective amount of purified donor muscle SP cells to the mammal. In a third embodiment, the invention relates to a method of treating BMD in a mammal in need thereof comprising administering an effective amount of purified donor muscle SP cells to the mammal. In the latter two embodiments, a proportion of the administered donor muscle SP cells can fuse with DMD or BMD host muscle fibers, contributing dystrophin-competent myonuclei to the host fibers (mosaic fibers). The expression of normal (donor) dystrophin genes in such fibers can generate sufficient dystrophin in some segments to confer a normal phenotype to these muscle fiber segments.

The invention also relates to a method of treating a limb-girdle muscular dystrophy in a mammal in need thereof comprising administering an effective amount of purified donor muscle SP cells to the mammal.

Muscle SP cells purified or isolated in accordance with the methods of the present invention can also be used in gene therapy, a utility enhanced by the ability of the muscle SP cells to proliferate and fuse. Muscle SP cells can be genetically altered by one of several means known in the art to comprise functional genes which may be defective or lacking in a mammal requiring such therapy. The recombinant muscle SP cells can then be transferred to a mammal, wherein they will multiply and fuse and, additionally, express recombinant genes. Using this technique, a missing or defective gene in a mammal's muscular system can be supplemented or replaced by infusion of genetically altered muscle SP cells. Gene therapy using muscle SP cells can also be applied in providing essential gene products through secretion from muscle tissue to the bloodstream (circulation). Because muscle SP cells proliferate and fuse together, they are capable of contributing progeny comprising recombinant genes to multiple, multinucleated myofibers during the course of normal muscular development.

Thus, muscle SP cells purified or isolated in accordance with the methods of the present invention can be used for delivery of a desired nucleic acid product to the circulation of a mammal (e.g., a human or other mammal or vertebrate). In this method, a nucleic acid sequence encoding a desired nucleic acid product is introduced into purified muscle SP cells. Typically, the nucleic acid sequence will be a gene which encodes the desired nucleic acid product. Such a gene is typically operably linked to suitable control sequences capable of effecting the expression of the desired nucleic acid product in muscle SP cells. The term “operably linked”, as used herein, is defined to mean that the gene (or the nucleic acid sequence) is linked to control sequences in a manner which allows expression of the gene (or the nucleic acid sequence). Generally, operably linked means contiguous.

Control sequences include a transcriptional promoter, an optional operator sequence to control transcription, a sequence encoding suitable mRNA ribosomal binding sites and sequences which control termination of transcription and translation. Suitable control sequences also include myoblast-specific transcriptional control sequences (see, e.g., U.S. Pat. No. 5,681,735, the teachings of which are incorporated herein by reference). Thus, in a particular embodiment, a recombinant gene (or a nucleic acid sequence) encoding a desired nucleic acid product is operably linked to myoblast-specific control sequences capable of effecting the expression of the desired nucleic acid product in muscle SP cells. In a further embodiment, a nucleic acid sequence encoding a desired nucleic acid product can be placed under the regulatory control of a promoter which can be induced or repressed, thereby offering a greater degree of control with respect to the level of the product in the muscle SP cells.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known in the art. Exemplary promoters include the SV40 and human elongation factor (EFI). Other suitable promoters are readily available in the art (see, e.g., Ausubel et al., Current Protocols in Molecular Biology, John Wiley & Sons, Inc., New York (1998); Sambrook et al., Molecular Cloning: A Laboratory Manual, 2nd edition, Cold Spring Harbor University Press, New York (1989); and U.S. Pat. No. 5,681,735).

Nucleic acid sequences are defined herein as heteropolymers of nucleic acid molecules. The nucleic acid molecules can be double stranded or single stranded and can be a deoxyribonucleotide (DNA) molecule, such as cDNA or genomic DNA, or a ribonucleotide (RNA) molecule. As such, the nucleic acid sequence can, for example, include one or more exons, with or without, as appropriate, introns, as well as one or more suitable control sequences. In one example, the nucleic acid molecule contains a single open reading frame which encodes a desired nucleic acid product. The nucleic acid sequence is operably linked to a suitable promoter.

A nucleic acid sequence encoding a desired nucleic acid product can be isolated from nature, modified from native sequences or manufactured de novo, as described in, for example, Ausubel et al., Current Protocols in Molecular Biology, John Wiley & Sons, New York (1998); and Sambrook et al., Molecular Cloning: A Laboratory Manual, 2nd edition, Cold Spring Harbor University Press, New York. (1989). Nucleic acids can be isolated and fused together by methods known in the art, such as exploiting and manufacturing compatible cloning or restriction sites.

As used herein, the term “desired nucleic acid product” refers to a protein or polypeptide, DNA (e.g., genes, antisense DNA) or RNA (e.g., ribozymes) to be expressed in a mammal. In a particular embodiment, the desired nucleic acid product is a heterologous therapeutic protein. Examples of therapeutic proteins include antigens or immunogens, such as a polyvalent vaccine, cytokines, tumor necrosis factor, interferons, interleukins, adenosine deaminase, insulin, T-cell receptors, soluble CD4, growth factors, such as epidermal growth factor, human growth factor, insulin-like growth factors, fibroblast growth factors), blood factors, such as Factor VIII, Factor IX, cytochrome b, glucocerebrosidase, ApoE, ApoC, ApoAI, the LDL receptor, negative selection markers or “suicide proteins”, such as thymidine kinase (including the HSV, CMV, VZV TK), anti-angiogenic factors, Fc receptors, plasminogen activators, such as t-PA, u-PA and streptokinase, dopamine, MHC, tumor suppressor genes such as p53 and Rb, monoclonal antibodies or antigen binding fragments thereof, drug resistance genes, ion channels, such as a calcium channel or a potassium channel, adrenergic receptors, hormones (including growth hormones) and anti-cancer agents. In another embodiment, the desired nucleic acid product is a gene product to be expressed in a mammal and which product is otherwise defective or absent in the mammal.

For example, in the treatment of a mammal with DMD or BMD, the desired nucleic acid product can be dystrophin. In the treatment of a mammal with a limb-girdle muscular dystrophy, desired nucleic acid products include, but are not limited to, calpain-3 and sarcoglycan complex members (e.g., α-sarcoglycan, β-sarcoglycan, γ-sarcoglycan and δ-sarcoglycan). In the treatment of a mammal with a congenital muscular dystrophy, desired nucleic acid products include, but are not limited to, laminin α2-chain. In the treatment of a mammal with cancer, desired nucleic acid products include, but are not limited to, anticancer agents.

Nucleic acid sequences encoding a desired nucleic acid product can be introduced into purified muscle SP cells by a variety of methods (e.g., transfection, infection, transformation, direct uptake, projectile bombardment, using liposomes). In a particular embodiment, a nucleic acid sequence encoding a desired nucleic acid product is inserted into a nucleic acid vector, e.g., a DNA plasmid, virus or other suitable replicon (e.g., viral vector). As a particular example, a nucleic acid sequence encoding a desired nucleic acid product is integrated into the genome of a virus which is subsequently introduced into purified muscle SP cells. The term “integrated”, as used herein, refers to the insertion of a nucleic acid sequence (e.g., a DNA or RNA sequence) into the genome of a virus as a region which is covalently limked on either side to the native sequences of the virus. Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

Packaging cell lines can be used for generating recombinant viral vectors comprising a recombinant genome which includes a nucleotide sequence (RNA or DNA) encoding a desired nucleic acid product. The use of packaging cell lines can increase both the efficiency and the spectrum of infectivity of the produced recombinant virions.

Packaging cell lines useful for generating recombinant viral vectors comprising a recombinant genome which includes a nucleotide sequence encoding a desired nucleic acid product are produced by transfecting host cells, such as mammalian host cells, with a viral vector including the nucleic acid sequence encoding the desired nucleic acid product integrated into the genome of the virus, as described herein. Suitable host cells for generating cell lines include human (such as HeLa cells), bovine, ovine, porcine, murine (such as embryonic stem cells), rabbit and monkey (such as COS1 cells) cells. A suitable host cell for generating a cell line may be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions). These cells can be obtained commercially or from a depository or obtained directly from an individual, such as by biopsy. Viral stocks are harvested according to methods generally known in the art. See, e.g., Ausubel et al., Eds., Current Protocols In Molecular Biology, John Wiley & Sons, New York (1998); Sambrook et al., Eds., Molecular Cloning: A Laboratory Manual, 2nd edition, Cold Spring Harbor University Press, New York (1989); Danos and Mulligan, U.S. Pat. No. 5,449,614; and Mulligan and Wilson, U.S. Pat. No. 5,460,959, the teachings of which are incorporated herein by reference.

Examples of suitable methods of transfecting or transforming muscle SP cells include infection, calcium phosphate precipitation, electroporation, microinjection, lipofection and direct uptake. Such methods are described in more detail, for example, in Sambrook et al., Molecular Cloning: A Laboratory Manual, Second Edition, Cold Spring Harbor University Press, New York (1989); Ausubel, et al., Current Protocols in Molecular Biology, John Wiley & Sons, New York (1998); and Danos and Mulligan, U.S. Pat. No. 5,449,614, the teachings of which are incorporated herein by reference.

Virus stocks consisting of recombinant viral vectors comprising a recombinant genome which includes a nucleotide (DNA or RNA) sequence encoding a desired nucleic acid product, are produced by maintaining the transfected cells under conditions suitable for virus production (e.g., in an appropriate growth media and for an appropriate period of time). Such conditions, which are not critical to the invention, are generally known in the art. See, e.g., Sambrook et al., Molecular Cloning: A Laboratory Manual, Second Edition, Cold Spring Harbor University Press, New York (1989); Ausubel et al., Current Protocols in Molecular Biology, John Wiley & Sons, New York (1998); U.S. Pat. No. 5,449,614; and U.S. Pat. No. 5,460,959, the teachings of which are incorporated herein by reference.

A vector comprising a nucleic acid sequence encoding a desired nucleic acid product can also be introduced into muscle SP cells by targeting the vector to cell membrane phospholipids. For example, targeting of a vector can be accomplished by linking the vector molecule to a VSV-G protein, a viral protein with affinity for all cell membrane phospholipids. Such a construct can be produced using methods well known to those practiced in the art.

As a particular example of the above approach, a recombinant gene (or a nucleic acid sequence) encoding a desired nucleic acid product and which is operably linked to myoblast-specific control sequences capable of effecting the expression of the desired nucleic acid product in purified muscle SP cells can be integrated into the genome of a virus that enters the SP cells. By infection, the muscle SP cells can be genetically altered to comprise a stably incorporated recombinant gene (or a nucleic acid sequence) encoding a desired nucleic acid product and which is under myoblast-specific transcription control. Muscle SP cells genetically altered in this way (recombinant muscle SP cells) can then be examined for expression of the recombinant gene (or nucleic acid sequence) prior to administration to a mammal. For example, the amount of desired nucleic acid product expressed can be measured according to standard methods (e.g., by immunoprecipitation). In this manner, it can be determined in vitro whether a desired nucleic acid product is capable of expression to a suitable level (desired amount) in the muscle SP cells prior to administration to a mammal. Genetically altered muscle SP cells (recombinant muscle SP cells) expressing the desired nucleic acid product to a suitable level can be expanded (grown) for introduction into the circulation of a mammal. Methods for expanding (growing) cells are well known in the art. As discussed above, in a particular embodiment, muscle SP cells are purified from a donor matched for immunocompatibility with the recipient mammal. Preferably, the donor and recipient are matched for their compatibility for the MHC (HLA)-class I (A, B, C) and -class II (DR, DQ, DRW) antigens.

The present invention further relates to a method of transplanting bone marrow in a mammal in need thereof (e.g., an irradiated individual or an individual undergoing chemotherapy) comprising introducing into the mammal purified muscle SP cells. Purified muscle SP cells of the present invention can be used to treat diseases or conditions in which a mammal needs bone marrow cells. Such diseases and conditions include, but are not limited to, leukemia, thalassemia and anemia.

Purified muscle SP cells, either genetically altered as described herein or unaltered, can be administered to (introduced into) a mammal according to methods known to those practiced in the art. Preferably, the mode of administration is systemically by injection. Other modes of administration (parenteral, mucosal, implant, intraperitoneal, intradermal, transdermal (e.g., in slow release polymers), intramuscular, intravenous including infusion and/or bolus injection, subcutaneous) are generally known in the art. Preferably, muscle SP cells are administered in a medium suitable for injection into a mammal, such as phosphate buffered saline.

The purified muscle SP cells used in the methods of the present invention can be obtained from a mammal to whom they will be returned or from another/different mammal of the same or different species (donor) and introduced into a recipient mammal. For example, the cells can be obtained from a pig and introduced into a human. In an embodiment of particular interest, the recipient mammal is a human patient.

An “effective amount” of purified muscle SP cells is defined herein as that amount of muscle SP cells which, when administered to a mammal, is sufficient for therapeutic efficacy (e.g., results in clinical improvement) (e.g., an amount sufficient for significantly reducing or eliminating symptoms and/or signs associated with the disease of interest). For example, in the case of a mammal with cancer or leukemia or other genetic disease that does not affect the muscle (e.g., a mammal with an inherited or acquired disease other than a muscle disease), an effective amount of purified muscle SP cells is that amount of muscle SP cells, which when administered to the mammal, can differentiate in bone marrow. In the case of a mammal with a muscle disease, an effective amount of purified muscle SP cells is that amount of muscle SP cells, which when administered to the mammal, can proliferate and fuse together to form mature muscle fibers. The amount of donor muscle SP cells administered to a mammal, including frequency of administration, will vary depending upon a variety of factors, including mode and route of administration; size, age, sex, health, body weight and diet of the recipient; the disease or disorder being treated; the nature and extent of symptoms of the disease or disorder being treated; kind of concurrent treatment, frequency of treatment, and the effect desired.

The present invention will now be illustrated by the following examples, which are not to be considered limiting in any way.

### EXAMPLES

The following methods were used in Examples 1, 2, 3 and 4.

### Animals

The X-linked muscular dystrophic (mdx) mouse is an animal model of Duchenne muscular dystrophy (DMD) (Bulfield, G. et al., Proc. Natl. Acad. Sci. USA, 81:1189-1192 (1984); and Sicinski, P. et al., Science, 244:1578-1580 (1989)) and serves as a good approximation to the human disease. The mdx mouse has the same genetic defect as occurs in DMD. As in DMD, its muscle fibers lack the protein dystrophin (Hoffman, E. P. et al., Cell, 51:919-928 (1987)) and undergo widespread degeneration.

Normal donor male mice (C57BL10) and recipient mdx female mice (C57BL10 dmd/dmd) used in the studies described herein were purchased from the Jackson Laboratory (Bar Harbor, Me.). Animals were maintained according to institutional guidelines.

Recipient 4-6 weeks old mdx females were lethally irradiated with 1,100 rads using a cesium source. The radiation was administered in split doses at least 2 hours apart. After cell injections, recipient animals were maintained on acidified water to prevent infections.

### Isolation of Murine Muscle SP Cells

Skeletal muscle myoblasts were isolated from skeletal muscle harvested from the hind legs of five 3-5 week old male mice as previously described (Rando, T. A. and Blau, H. M., J. Cell Biol., 125:1275-1287 (1994)). Prior to H0342 staining, red cells were lysed (Baroffio, A. et al., Differentiation, 59:259-268 (1995)). Cells were then resuspended at 10⁶ cells/ml and stained with 12.5 μg/ml of H0342 in PBS-0.5% BSA for 90 minutes at 37° C. In parallel, 10⁶ cells were stained as described in the presence of 50 μM verapamil (Goodell, M. A. et al., J. Exp. Med., 183:1797-1806 (1996)). Samples stained in the presence of verapamil were used as a negative control for SP cells, and were utilized to set the gate for isolation of SP cells by FACS in the test sample. Cells were washed once in cold PBS-0.5% BSA, resuspended at 10⁸ cells/ml and incubated for 10 minutes on ice with 10 μg/ml of biotinylated anti-Sca-1 antibody (Pharamingen).

Prior to FACS analysis and sorting, cells were enriched for Sca-1⁺ cells using the MACS columns (Miltenyi Biotech, Sunnyvale, Calif.) and stained with 2 μg/ml of propidium iodide (PI) (Goodell, M. A. et al., J. Exp. Med., 183:1797-1806 (1996)).

For cell lineage marker analysis, muscle SP and MP cells were stained with different cell lineage marker antibodies as previously described (Goodell, M. A. et al., J. Exp. Med., 183:1797-1806 (1996)). Cells were analyzed and isolated using a dual-laser FACS Vantage flow cytometer (Becton Dickinson) as previously described (Goodell, M. A. et al., J. Exp. Med., 183:1797-1806 (1996)).

Prior to injections into animals, muscle SP cells were washed once in PBS-0.5% BSA and resuspended in 200 μl of PBS-0.5% BSA. For cell culture, muscle SP cells and MP myoblasts were resuspended in Ham's F10 supplemented with 20% fetal bovine serum and 10 ng/ml bFGF (Promega), as previously described (Rando, T. A. and Blau, H. M., J. Cell Biol., 125:1275-1287 (1994)). Cells were plated on tissue culture plates coated with E-C-L (Upstate Biotechnology) and maintained at 37° C. in a humidified chamber with 5% CO₂.

### Tissue Collection and Fluorescent In Situ Hybridization Analysis

Recipient animals were euthanized according to institutional guidelines, and skeletal muscle and spleen were snap-frozen in cold isopentane and stored at −80° C. The bone marrow was isolated from the hind leg bones using a mortar and pestle. Cells were washed in PBS and filtered through a 70 μM filter.

For Giemsa staining, bone marrow cells were spread on a glass slide and fixed in methanol for 3 minutes. Cells were stained in Giemsa stain (Sigma) according to the manufacturer instructions.

For fluorescent in situ hybridization (FISH) analysis of bone marrow nuclei, cells were treated with hypotonic solution and fixed in methanol and acetic acid prior to slide preparation as previously described (Buckle, V. J. and Kearney, L., Current Opin. Genetics Develop., 4:374-382 (1994); and Lichter, P. et al., Proc. Natl. Acad. Sci. USA, 85:9664-9668 (1988)).

The Y-chromosome FISH probe (a generous gift of Dr. E. Snyder) (see also, e.g., Nishioka, Y., Teratology, 38:181-185 (1988); Harvey, A. R. et al., Brain Res. Mol. Brain Res., 12:339-343 (1992); Prado, V. F. et al., Cytogenet. Cell Genet., 61:87-90 (1992); and Harvey, A. R. et al., Int. J. Dev. Neurosci., 11:569-581 (1993)) was prepared by labeling one microgram of plasmid DNA with digoxigenin-11-dUTP as previously described (Gussoni, E. et al., Nat. Biotechnol., 14:1012-1016 (1996); and Lichter, P. et al., Proc. Natl. Acad. Sci. USA, 85:9664-9668 (1988)). FISH was standardized on whole nuclei isolated from a male murine muscle cell line and on male muscle tissue sections. The hybridization efficiency was greater than 90% on whole nuclei and 84% on tissue sections.

Immunohistochemistry and in situ hybridization were preformed on the same tissue sections as previously described (Gussoni, E. et al., Nat. Biotechnol., 14:1012-1016 (1996)). Nuclei were counterstained with 4′-6′ diamidino-2-phenylindole (DAPI) (200 ng/ml), and slides were examined using a Zeiss Axiophot microscope. Visual inspection of the results through a triple band-pass filter (Omega, Brattleboro, Vt.) revealed the preservation of the protein signal by immunohistochemistry and the simultaneous detection of the DNA hybridization signal over the DAPI counterstained nuclei. Images were collected from the same microscopic field using a CCD camera (Photometrics, Tucson, Ariz.) as previously described (Gussoni, E. et al., Nat. Biotechnol., 14:1012-1016 (1996)).

### Example 1

### Identification of Muscle SP Cells and Muscle MP Cells

Mononuclear cells were isolated from 3-5 week old male mouse skeletal muscle (Rando, T. A. and Blau, H. M., J. Cell Biol., 125:1275-1287 (1994)) and stained with 12.5 μg/ml of H0342 and 2 μg/ml propidium iodine (PI). FACS analysis of the cells revealed a side population (SP) displaying low staining with H0342 and a main population (MP) of cells more brightly stained with the dye. PI fluorescence was also measured.

Similar to what was seen for bone marrow SP cells, the muscle SP cell population disappeared upon the addition of verapamil, a drug that blocks the efflux of Hoechst dye by inhibiting a multidrug resistance-like protein presumed to be expressed by SP cells (Goodell, M. A. et al., J. Exp. Med., 183:1797-1806 (1996)). In contrast, the MP myoblasts were unaffected by verapamil. These results indicate the identification of a population of cells from skeletal muscle (muscle SP) that are potentially similar to the previously described bone marrow SP stem cells (Goodell, M. A. et al., J. Exp. Med., 183:1797-1806 (1996)).

### Example 2

### Characterization of Muscle SP Cells and Muscle MP Cells

Characterization of muscle SP cells revealed several unique features that distinguished them from bone marrow SP cells. First, isolation of muscle SP cells required a concentration of H0342 dye that was 2.5 times greater than that used to purify bone marrow SP cells (Goodell, M. A. et al., J. Exp. Med., 183:1797-1806 (1996)). This amount of dye is lethal to the vast majority of bone marrow SP cells.

Expression of cell surface antigens on muscle SP and MP cells was studied using the FACS. Over 80% of muscle SP cells express the antigen Sca-1 and are negative for lineage markers. In contrast to bone marrow SP cells, the majority of muscle SP cells are negative for CD43, c-kit and CD45. Thus, muscle and bone marrow SP cells differed in their expression of cell surface markers. That is, although both muscle and bone marrow SP cells were Sca-1⁺ lin⁻, as predicted for early progenitor cells, c-kit and CD45, two surface marker expressed on bone marrow SP cells (Goodell, M. A. et al., Nat. Med., 3:1337-1345 (1997)), were not present of muscle SP cells. Similarly, over 90% of muscle SP cells were negative for CD43, another marker detected on bone marrow SP cells (Goodell, M. A. et al., Nat. Med., 3:1337-1345 (1997)). These results imply that muscle and bone marrow SP cells consist of two distinct cell populations that express different patterns of surface antigens. In contrast, muscle MP cells appeared to be more differentiated than muscle SP cells, since the MP cells expressed some lineage markers (lin⁺), such as CD11, Gr-1 and CD5. A summary of the antigens expressed on muscle SP and MP cells compared to bone marrow SP cells is reported in Table 1.

TABLE 1
Summary of Antigens Expressed On Murine Muscle SP and MP Cells.

| | CD34 | Sca-1 | CD43 | c-kit | CD11 | CD45 | Gr-1 | B220 | CD5 | CD4/CD8 |
|---|---|---|---|---|---|---|---|---|---|---|
| Muscle | +/− | +/− | +/− | − | +/− | − | + | − | +/− | −/− |
| MP | | | | | | | | | | |
| Muscle | +/− | + | − | − | − | − | − | − | − | −/− |
| SP | | | | | | | | | | |
| Bone | − | + | + | + | − | + | − | − | − | −/− |
| Marrow | | | | | | | | | | |
| SP | | | | | | | | | | |

+/−: mixture of positive and negative cells;
+: cells positive for the marker;
−: cells negative for the marker.

To further analyze their characteristics, muscle SP and MP cells were cultured in vitro. After one week, nearly all MP cells adhered to the culture dish and were fully differentiated into myoblasts, with a few intervening fibroblasts. In contrast, most SP cells maintained a spherical shape and failed to settle on the plate. Only after 2 weeks in culture did muscle SP cells differentiate as a mixture of myoblasts and fibroblasts. Sequential cloning of muscle SP cells transduced with a v-myc retrovirus indicated that single colonies of muscle SP clones maintained the ability to differentiate into myoblasts and fibroblasts in vitro. Thus, muscle SP cells appear to be derived from a less differentiated progenitor population than the MP cells.

### Example 3

### Ability of Muscle SP Cells to Differentiate into Mesodermal-Derived Cell Types In Vivo

Because initial characterization indicated that muscle SP cells are mesodermal precursors with some similarities to the previously described bone marrow SP cells, the potential of muscle SP cells to differentiate into diverse mesodermal-derived cell types in vivo was investigated.

Muscle SP and MP cells were prepared from normal C57BL/10 male mice and injected into the tail veins of lethally irradiated female mdx mice. Introduced donor cells were detected in host (recipient) tissues (e.g., skeletal muscle, bone marrow, spleen, heart, liver) using fluorescence in situ hybridization (FISH) to demonstrate the presence of Y-chromosomal DNA (Grounds, M. D. et al., Transplantation, 52:1101-1105 (1991)). Four lethally irradiated host animals were injected with an equal number of either muscle MP or SP cells (4,000 or 10,000 cells) per animal. Animals injected with MP cell population died approximately 10-12 days after cell injection. Similarly, an animal injected with 4,000 muscle SP cells died within 2 weeks after cell injection. In contrast, one mouse injected with 10,000 muscle SP cells appeared in good health when it was sacrificed at 3 weeks after cell injection (Table 2, animal 1). These results suggest that unlike muscle MP cells, muscle SP cells can differentiate into bone marrow and protect animals from the effects of lethal irradiation.

TABLE 2
Injection of Donor Male Muscle SP Cells Into mdx Females.

| | | | Avg. # of | | |
|---|---|---|---|---|---|
| mdx | Age (days) | Number of | Dystrophin | Avg. # | % Y Nuclei |
| Recipient | Recipient | Muscle SP Cells | Positive | of Y | in Bone |
| Animal | Euthanized¹ | Injected | Myofibers² | Nuclei³ | Marrow⁴ |
| 1 | 21 | 10,000 | 13 | 1 | 75 |
| 2 | 17 | 7,000 | 27 | 1 | 41 |
| 3 | 30 | 20,000 | 47 (32) | 2 (3) | 91 |
| 4 | 30 | 13,000 | 15 | 1 | 30 |
| 5 | 28 | 19,000 | ND | ND | 80 |

¹Age of the mdx recipient at the time it was euthanized.
²Average number of dystrophin positive myofibers in skeletal muscle tissue sections. For each animal, between 15-30 sections were analyzed. For animal 2, numbers are given for 2 separate experiments.
³Average number of Y nuclei fused to dystrophin positive myofibers in an individual tissue section.
⁴Percentage of Y nuclei in the bone marrow samples detected by FISH. As a positive control for these experiments, the FISH probe was hybridized in parallel to interphase nuclei from male cells, and the hybridization efficiency was over 95%.
ND = Not Determined

To further study this possibility, 7,000-20,000 male muscle SP cells were injected into 4 additional lethally irradiated mdx females (Table 2). At day 17, one mouse injected with 7,000 muscle SP cells (Table 2, animal 2) appeared weak and was sacrificed. The other animals were euthanized at 28-30 days and all seemed in good health at the time of sacrifice (Table 2, animals 3-5). Skeletal muscle, bone marrow and spleen of these animals were collected for analysis.

To investigate whether muscle SP cells could differentiate into mature muscle, skeletal muscle tissue sections of 4 animals injected with muscle SP cells were analyzed by immunohistochemistry combined with FISH, as previously described (Gussoni, E. et al., Nat. Biotechnol., 14:1012-1016 (1996); and Gussoni, E. et al., Nat. Med., 3:970-977 (1997)) (Table 2). In each section, dystrophin-positive myofibers were detected by immunohistochemistry, and FISH analysis of the same tissue sections revealed the presence of donor male nuclei fused to the dystrophin-producing host myofibers, at least one per section. In 18 photographed muscle tissue sections from 3 different animals, a total of 28 donor male nuclei were detected. Twelve of these nuclei were centrally located in the dystrophin-positive myofibers, 9 were peripherally located, and 7 were clearly fused to the myofibers but whether they were centrally or peripherally located was unclear. Further analysis of these tissue sections revealed a few donor nuclei juxtaposed to a myofiber, a feature characteristic of satellite cells (Mauro, A., J. Biophys. Biochem. Cytol., 9:493-495 (1961); Bischoff, R., in Myology, Engel, A. G. and Franzini-Armstrong, C., Eds., New York: Mc Graw Hill, pp. 97-119 (1994); and Grounds, M., Adv. Exp. Med. & Biol., 280:101-104 (1990)), which suggests that a portion of donor muscle SP cells have the ability to divide and contribute to muscle regeneration (Gussoni, E. et al., Nat. Med., 3:970-977 (1997); and Yao, S. N. and Kurachi, K., J. Cell Sci., 105:957-963 (1993)). These results indicate that muscle SP cells delivered systematically are successfully recruited from the circulation into skeletal muscles, where they are able to fuse with pre-existing myofibers and express dystrophin.

Analysis of dystrophin positive fibers within the entire field of muscle tissue sections indicated that up to 9% of mdx host myofibers produced dystrophin after systemic injection of muscle SP cells. Although not all dystrophin-positive myofibers in a given tissue section showed fused donor nuclei (Table 2), studies of nuclear domains in myofibers have shown that the expression of a protein can extend several microns away from the source nucleus (Karpati, G. et al., Am. J. Pathol., 135:27-32 (1989); Gussoni, E. et al., Nat. Med., 3:970-977 (1997); Hall, Z. W. and Ralston, E. A., Cell, 59:771-772 (1989); and Pavlath, G. K. et al., Nature, 337:570-573 (1989)). Alternatively, a few of these dystrophin-positive fibers might have been revertant fibers (Hoffman, E. P. et al., J. Neurol. Sci., 99:9-25 (1990)). However, analysis of skeletal muscle tissue sections of control irradiated animals revealed 0-4 revertant fibers per section (0-0.8%), a number substantially less than the 13-47 positive fibers detected in muscle SP-injected animals (Table 2).

Because muscle SP cells appeared capable of protecting animals from the effects of lethal irradiation, bone marrow samples of the mdx mice injected with muscle SP cells were analyzed by FISH (Table 2). Between 30%-91% of the bone marrow nuclei showed hybridization with the Y-chromosome probe, proving that they were of donor origin (Table 2). At high magnification (100×), metaphase spreads containing the Y-chromosome were also detected in the bone marrow samples of animals 3 and 4 by FISH, indicating that the introduced male muscle SP cells are capable of dividing in vivo. Giemsa staining of bone marrow samples of these animals revealed the presence of diverse types of hematopoietic cells.

In addition to bone marrow, spleen, another hematopoietic organ, was analyzed for the presence of donor cells. Spleen tissue sections of animals 3 and 5 were immunostained with either anti-CD43 or anti-CD45 antibodies, two surface markers expressed by hematopoietic cells but not by muscle SP cells (Table 1). Both antibodies revealed the presence of immunoreactive cells. Codetection of donor nuclei by FISH demonstrated that over 90% of the spleen cells, including the ones expressing CD43 or CD45, were positive for the Y-chromosome and thus of donor origin. These observations indicate that muscle SP cells can differentiate into bone marrow and spleen tissues, and are able to protect animals from the consequences of lethal irradiation.

### Example 4

### Comparison of the Efficacy of Muscle SP Cells Versus Bone Marrow SP Cells to Reconstitute Bone Marrow

To compare the efficacy of muscle SP versus previously described bone marrow SP cells (Goodell, M. A. et al., J. Exp. Med., 183:1797-1806 (1996); and International Publication No. WO 9639489, published Dec. 12, 1996) to reconstitute the bone marrow, lethally irradiated mdx females were injected with a mixture of 200 bone marrow SP cells derived from mdx females and 6,000 muscle SP cells derived from normal male mice. In these experiments, animals were sacrificed at 4 and 8 weeks, and analyzed for the presence of donor male nuclei in the bone marrow. Less than 1% of the nuclei were positive for the Y-chromosome by FISH. These data imply that, in a competitive experiment, bone marrow SP cells are more efficient at repopulating the bone marrow of lethally irradiated animals than muscle SP cells, though there is a residual contribution by muscle SP cells as indicated by the presence of a few male donor nuclei.

### Example 5

### Isolation and Characterization of Human Muscle SP Cells and Human Muscle MP Cells

Muscle myoblasts were isolated from human muscle tissue obtained from muscle biopsies using standard methods (see, e.g., Blau, H. M. et al., Adv. Exp. Med. Biol., 280:97-100 (1990); Blau, H. M. et al., Proc. Natl. Acad. Sci. USA, 78:5623-5627 (1981); Rando, T. A. and Blau, H. M., J. Cell. Biol., 125:1275-1287 (1994)). Prior to H0342 staining, red cells were lysed (Baroffio, A. et al., Differentiation, 59:259-268 (1995)). Cells were then resuspended at 10⁶ cells/ml and stained with 1-5 μg/ml of H0342 in PBS-0.5% BSA for 60 minutes at 37° C. In parallel, 10⁶ cells were stained as described in the presence of 100 μM verapamil (Goodell, M. A. et al., J. Exp. Med., 183:1797-1806 (1996)) or in the presence of 10 μM PAK-104P (Chen, Z. S. et al., Molecular Pharmacology, 55:921-928 (1999); Marbeuf-Gueye, C. et al., Eur. J. Pharmcol., 391:207-216 (2000)). Samples stained in the presence of verapamil or PAK-104P were used as a negative control for SP cells, and were utilized to set the gate for isolation of SP cells by FACS in the test sample.

Expression of cell surface antigens on muscle SP and MP cells was studied using FACS. Muscle SP cells were negative for CD 123. A summary of the antigens expressed on human muscle SP and MP cells is reported in Table 3.

TABLE 3
Summary of Antigens Expressed on Human Muscle SP and MP Cells.

| | CD34 | CD90 | c-kit | CD123 | AC133 |
|---|---|---|---|---|---|
| Muscle MP | +/− | +/− | − | +/− | +/− |
| Muscle SP | +/− | +/− | − | − | +/− |

+/−: mixture of positive and negative cells;
−: cells negative for the marker.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method for delivery of a muscle protein to the circulation of a mammal comprising administering purified donor muscle side population cells to said mammal.

2. A method of claim 1 wherein said muscle protein is dystrophin.

3. A method of claim 1 wherein said animal is human.

4. A method of transplanting bone marrow in a mammal comprising introducing purified donor muscle side population cells into said mammal.

5. A method of claim 4 wherein said mammal is human.

6. A method for delivery of a desired nucleic acid product to the circulation of a mammal comprising the steps of:
a) introducing a nucleic acid sequence encoding said desired nucleic acid product into muscle side population cells, whereby recombinant muscle side population cells are produced; and
b) administering to said mammal recombinant muscle side population cells produced in step a),
whereby said nucleic acid product is delivered to the circulation of said mammal.

7. A method of claim 6 wherein said muscle side population cells are obtained from said mammal into which said cells are to be delivered.

8. A method of claim 6 wherein said muscle side population cells are obtained from a donor mammal.

9. A method of claim 6 wherein said desired nucleic acid product is a heterologous therapeutic protein.

10. A method of claim 6 wherein said nucleic acid sequence is incorporated into a viral vector.

11. A method of claim 6 wherein said mammal is human.

12. A method of treating a muscle disease in a mammal in need thereof comprising administering an effective amount of purified donor muscle side population cells to said mammal.

13. A method of claim 12 wherein said muscle disease is a muscular dystrophy.

14. A method of claim 13 wherein said muscular dystrophy is selected from the group consisting of: Duchenne muscular dystrophy and Becker muscular dystrophy.

15. A method of claim 13 wherein said muscular dystrophy is a limb girdle muscular dystrophy.

16. A method of claim 12 wherein said mammal is human.

17. A method of treating a muscle disease in a mammal in need thereof comprising the steps of:
a) introducing a nucleic acid sequence encoding a desired nucleic acid product into muscle side population cells, whereby recombinant muscle side population cells are produced; and
b) administering recombinant muscle stem cells produced in step a) to said mammal.

18. A method of claim 17 wherein said nucleic acid sequence is incorporated into a viral vector.

19. A method of claim 17 wherein said muscle side population cells are obtained from said mammal to be treated.

20. A method of claim 17 wherein said muscle side population cells are obtained from a donor mammal.

21. A method of claim 17 wherein said muscle disease is a muscular dystrophy.

22. A method of claim 21 wherein said muscular dystrophy is selected from the group consisting of: Duchenne muscular dystrophy and Becker muscular dystrophy.

23. A method of claim 22 wherein said desired nucleic acid product is dystrophin.

24. A method of claim 21 wherein said muscular dystrophy is a limb girdle muscular dystrophy.

25. A method of claim 17 wherein said mammal is human.

26. Purified muscle side population cells that are c-kit^{neg} and CD45^{neg}.

27. Purified muscle side population cells of claim 26 that are CD43^{neg}.

28. A method of treating or prophylaxis of a cancer in a mammal in need thereof comprising the steps of:
a) introducing a nucleic acid sequence encoding a desired nucleic acid product into muscle side population cells, whereby recombinant muscle side population cells are produced; and
b) administering recombinant muscle side population cells produced in step a) to the mammal.

29. A method of claim 28 wherein said desired nucleic acid product is a heterologous therapeutic protein.

30. A method of claim 28 wherein said nucleic acid sequence is incorporated into a viral vector.

31. A method of claim 28 wherein said muscle side population cells are obtained from said mammal to be treated.

32. A method of claim 28 wherein said muscle side population cells are obtained from a donor mammal.

33. A method of claim 28 wherein said mammal is human.

34. A method of treating or prophylaxis of a genetic disease in a mammal in need thereof comprising the steps of:
a) introducing a nucleic acid sequence encoding a desired nucleic acid product into muscle side population cells, whereby recombinant muscle side population cells are produced; and
b) administering recombinant muscle side population cells produced in step a) to the mammal.

35. A method of claim 34 wherein said desired nucleic acid product is a heterologous therapeutic protein.

36. A method of claim 34 wherein said nucleic acid sequence is incorporated into a viral vector.

37. A method of claim 34 wherein said muscle side population cells are obtained from said mammal to be treated.

38. A method of claim 34 wherein said muscle side population cells are obtained from a donor mammal.

39. A method of claim 34 wherein said mammal is human.

40. A method of treating or prophylaxis of an inherited or acquired disease in a mammal in need thereof comprising the steps of:
a) introducing a nucleic acid sequence encoding a desired nucleic acid product into muscle side population cells, whereby recombinant muscle side population cells are produced; and
b) administering recombinant muscle side population cells produced in step a) to the mammal.

41. A method of claim 40 wherein said desired nucleic acid product is a heterologous therapeutic protein.

42. A method of claim 40 wherein said nucleic acid sequence is incorporated into a viral vector.

43. A method of claim 40 wherein said muscle side population cells are obtained from said mammal to be treated.

44. A method of claim 40 wherein said muscle side population cells are obtained from a donor mammal.

45. A method of claim 40 wherein said mammal is human.

46. A method of purifying muscle side population cells from a myoblast sample isolated from mammalian skeletal muscle comprising the steps of:
a) combining the myoblast sample with a fluorescent, lipophilic vital dye which is a substrate for a multiple drug resistant protein, under conditions appropriate for uptake of the dye by cells in the myoblast sample;
b) exposing the combination produced in step a) to an excitation wavelength which causes fluorescence of the dye;
c) assessing the fluorescence of the dye using an emission wavelength;
d) analyzing the amount of dye exhibited in each cell population; and
e) isolating the population of nucleated cells which contains the lowest amount of dye, thereby purifying muscle side population cells.

47. A method of claim 46 wherein said myoblast sample is isolated from human skeletal muscle.

48. A method of claim 46 wherein said dye is Hoechst 33342 dye, said excitation wavelength is about 350 nm and said emission wavelength is from about 600 nm to about 675 nm.

49. Purified muscle side population cells obtained according to a method of claim 46.

50. Purified muscle side population cells obtained according to a method of claim 47.

51. A method of separating muscle side population cells from muscle main population cells in a myoblast sample isolated from mammalian skeletal muscle comprising the steps of:
a) combining the myoblast sample with a fluorescent, lipophilic, vital dye which is a substrate for a multiple drug resistant protein under conditions appropriate for uptake of the dye by the cells in the myoblast sample;
b) exposing the combination produced in step a) to an excitation wavelength which causes fluorescence of the dye;
c) assessing the fluorescence of the dye using an emission wavelength;
d) analyzing the amount of dye exhibited in each cell population; and
e) isolating the population of nucleated cells which contains the lowest amount of dye from the population of nucleated cells which contains the greater amount of dye, thereby separating muscle side population cells from muscle main population cells.

52. A method of claim 51 wherein said myoblast sample is isolated from human skeletal muscle.

53. A method of claim 51 wherein said dye is Hoechst 33342 dye, said excitation wavelength is about 350 nm and said emission wavelength is from about 600 nm to about 675 nm.

54. Purified muscle side population cells obtained according to a method of claim 51.

55. Purified muscle side population cells obtained according to a method of claim 52.
